(19) United States
(12) Patent Application Publication
(10) Pub. No.: US 2014/0255364 A1
(43) Pub. Date: Sep. 11, 2014

(54) 3D CULTURE SYSTEM FOR CULTURING CELLS IN SYNOVIAL FLUID, CELLS CULTURED IN SYNOVIAL FLUID AND THEIR USE

(71) Applicant: TUFTS UNIVERSITY, Boston, MA (US)

(72) Inventors: Li Zeng, Framingham, MA (US); Joshua Brand, Boston, MA (US); Timothy McAlindon, Cambridge, MA (US)

(21) Appl. No.: 14/354,521

(22) PCT Filed: Oct. 26, 2012
(86) PCT No.: PCT/US2012/062294

§ 371 Date: Apr. 25, 2014

Related U.S. Application Data
(60) Provisional application No. 61/553,056, filed on Oct. 28, 2011.

(51) Int. Cl. A61K 35/32; G01N 33/50

(52) U.S. Cl. CPC A61K 35/32; G01N 33/5044
USPC 424/93.21; 435/404; 424/93.7; 435/29

(57) ABSTRACT

Disclosed herein are materials and methods for culturing cells derived from joint cavities, said culturing being performed in the presence of synovial fluid. Also disclosed herein are cells cultured by the methods described herein, and their use for therapeutic purposes and for identifying therapeutic agents.

### BACKGROUND

Arthritis, a disease that is caused by joint cartilage destruction, is the leading cause of disability in the United States. As cartilage in adult does not regenerate very efficiently in vivo, arthritis results in permanent cartilage loss, which is accompanied by chronic pain and immobility. Cartilage tissue engineering offers promising potential to regenerate articular cartilage in attempt to treat arthritis, however, most engineered cartilage is cultured in artificial media. In the native joint cavity, articular cartilage is naturally bathed in synovial fluid that consists of a wealth of growth factors, cytokines and enzymes that are important for chondrocyte metabolism.

Major hurdles in developing a more clinically relevant cultured chondrocyte for articular cartilage therapies have not been overcome. Any attempts to culture chondrocytes in a 3D environment surrounded by synovial fluid, for example, have encountered problems due to the high level of viscosity of synovial fluid. Several groups have supplemented synovial fluid to the culture medium in growing human, bovine, rabbit and dog chondrocytes, but these attempts have used only low level of synovial fluid (below 20%). While chicken, horse and human chondrocytes have been cultured in the medium with higher percentage of synovial fluid, such attempts have been made in 2D culture systems.

Due to the epidemic nature of osteoarthritis, there is high demand for improving technologies of cartilage regeneration. Such an improvement, for example, would be the development of cultured chondrocytes, cultured in such a way as to closely mimic their biological milieu. To best mimic the natural condition, engineered cartilage is best grown in 3D cultures in synovial fluid.

### SUMMARY

A three-dimensional (3D) system of culturing human articular chondrocytes in high levels of synovial fluid is described herein. Synovial fluid reflects the most natural microenvironment for articular cartilage, and can be easily obtained and stored. The system described herein can be used, for example, for studying cartilage regeneration, e.g., screening for factors that promote cartilage generation and for therapeutic agents that stimulate cartilage formation, generating therapeutic agents, e.g., cultured chondrocytes that can be, for example, injected into a subject at a point suitable to alleviate a disease or condition associated with chondrocyte or cartilage depletion, e.g., arthritis, and for screening therapeutics for treating arthritis.

In one embodiment, the disclosure is directed to a method of culturing mammalian cells that reside in a joint cavity in vivo, comprising: a) suspending one or more isolated cells in a three-dimensional matrix; and b) incubating the suspended cells in a culture medium comprising at least about 20% synovial fluid under conditions suitable for chondrocyte division. In a particular embodiment, the mammalian cells are selected from the group consisting of: chondrocytes, synoviocytes, meniscus cells and temporomandibular disc cells. In a particular embodiment, where chondrocytes are cultured, the chondrocytes are obtained commercially or derived directly from a mammalian subject, e.g., human. In a particular embodiment, the culture medium comprises at least about 20% synovial fluid, 30% synovial fluid, 40% synovial fluid, 50% synovial fluid, 60% synovial fluid, 70% synovial fluid, 80% synovial fluid, 90% synovial fluid or 100% synovial fluid. In a particular embodiment, the methods described herein further comprise one or more incubations in a culture medium comprising at least about 20% synovial fluid. In a particular embodiment, the culture medium is changed at least about every two or three days. In a particular embodiment, the three dimensional matrix comprises alginate. In a particular embodiment, the three dimensional makes forms a spherical bead about 2 mm in diameter. In a particular embodiment, the culture medium is supplemented with CaCl₂. In a particular embodiment, the methods described herein further comprise treating the suspended cells with sodium citrate. In a particular embodiment, the one or more suspended cells have been genetically modified.

In one embodiment, the disclosure is directed to a method of treating an injury, disease or disorder characterized by chondrocyte depletion comprising administering to a subject afflicted with an injury, disease or disorder characterized by chondrocyte depletion, an effective amount of cultured chondrocytes, wherein the chondrocytes are cultured in at least about 20% synovial fluid and wherein the chondrocytes are suspended in a three-dimensional matrix during culturing. In a particular embodiment, the synovial fluid used to culture the chondrocytes is derived from the subject afflicted with the disease or disorder. In a particular embodiment, the culture medium comprises at least about 20% synovial fluid, 30% synovial fluid, 40% synovial fluid, 50% synovial fluid, 60% synovial fluid, 70% synovial fluid, 80% synovial fluid, 90% synovial fluid or 100% synovial fluid. In a particular embodiment, the three dimensional matrix comprises alginate. In a particular embodiment, the three dimensional makes forms a spherical bead about 2 mm in diameter. In a particular embodiment, the method is used to treat osteoarthritis. In a particular embodiment, the cultured chondrocytes are removed from the three dimensional matrix prior to administering them to the subject. In a particular embodiment, the three-dimensional matrix comprises alginate. In a particular embodiment, the cultured chondrocytes are removed from the three dimensional matrix prior to administering them to the subject, said removal comprising washing the cells suspended in the matrix with sodium citrate. In a particular embodiment, the cultured chondrocytes administered to the subject have been genetically modified.

In one embodiment, the disclosure is directed to a mammalian cell, e.g., synoviocyte, meniscus cell, temporomandibular disc cell or chondrocyte, cultured by a method comprising: a) suspending one or more chondrocytes in a three-dimensional matrix; and b) incubating the suspended chondrocytes in a culture medium comprising at least about 20% synovial fluid under conditions suitable for chondrocyte division. In a particular embodiment, the one or more suspended chondrocytes have been genetically modified. In a particular embodiment, the three-dimensional matrix comprises alginate. In a particular embodiment, the chondrocyte is removed from the three-dimensional matrix by washing in sodium citrate.

In one embodiment, the disclosure is directed to a method of screening compounds for treating a disease or condition characterized by a depletion of cells in a joint cavity or loss of function of cells in a joint cavity, comprising: a) suspending one or more cells in a three-dimensional matrix, wherein the one or more cells are isolated from a subject with a disease or condition characterized by a depletion of cells in a joint cavity or loss of function of cells in a joint cavity; b) incubating the suspended cells in a culture medium comprising at least about 20% synovial fluid under conditions suitable for chondrocyte division in the presence and/or absence of a test compound; and c) determining a gene expression profile for one or more genes indicative of a disease state or condition, wherein a gene expression profile of the cells cultured in the presence of the test compound that is more similar to the gene expression profile of a healthy individual than to the gene expression profile of a subject afflicted with the disease or condition indicates the test compound is effective in treating the disease or condition. In a particular embodiment, the one or more isolated cells are chondrocytes. In a particular embodiment, the disease or condition is osteoarthritis.

### BRIEF DESCRIPTION OF THE DRAWINGS

FIG. 1 is a schematic diagram of a method to culture human articular chondrocytes in high percentages of synovial fluid in 3D alginate beads. Chondrocytes and alginate solution are mixed and then applied drop-wise to a CaCl₂ solution, wherein chondrocytes are immobilized within the crosslinked Ca²⁺-alginate hydrogel beads. These 3D constructs are then cultured in the chondrocyte differentiation medium (Lonza) with varying ratios of human synovial fluid (up to 100%). After 21 days of culturing under a rocking condition, alginate beads containing cells are washed extensively after which the cells are retrieved.

FIG. 2 are bright field (BF) and Dapi images of human articular chondrocytes encapsulated cultures with 0%, 30%, 50%, 70% and 100% synovial fluid. Chondrocytes were spherical in shape in all culture conditions. Images of bright field (BF) and Dapi were overlaid to confirm the location and distribution of chondrocytes. Insets: magnified images. Arrowheads: colocalization of chondrocytes in BF and Dapi staining images.

FIGS. 3A-D show qRT-PCR analysis of encapsulated human articular chondrocytes at day 0 (D0) an after 21 days of culturing (D21) in medium supplemented with varying ratios of synovial fluid (SF) (0%, 30%, 50%, 70% and 100%). Results of four independent samples are shown. GAPDH was used as internal reference for all PCRs. FIG. 3A shows collagen II mRNA expression. FIG. 3B shows aggrecan mRNA expression. FIG. 3C shows MMP13 mRNA expression. FIG. 3D shows caspase 3 mRNA expression. Statistical significance was assessed for D0 samples and D21 samples of 0% and 100% synovial fluid (SF) culturing, using INSTAT software. *denotes P<0.05.

### DETAILED DESCRIPTION

A three-dimensional (3D) culturing system is described herein suitable for the long-term culturing of human articular chondrocytes in high levels of synovial fluid (up to 100%). Conditions were discovered that allowed for the optimization of growth conditions, thereby overcoming hurdles presented by the high viscosity of the synovial fluid. Briefly, cells are suspended in a spherical matrix, e.g., created using alginate, and cultured while suspended in the spherical matrix. This is the first system that allows for the 3D growth of mammalian chondrocytes, e.g., human, in their natural milieu, synovial fluid. Synovial fluid is easy to obtain and store and can often be regularly replenished by the human body. The system described herein can be used to develop articular chondrocytes as a therapeutic agent, e.g., to be injected into a subject to replenish chondrocytes and/or cartilage, to screen for factors and therapeutic agents that stimulate or regulate chondrocyte growth and cartilage production, and to screen for therapeutic agents useful for treating conditions are diseases associated with chondrocyte or cartilage depletion, e.g., arthritis or sports injury. The terms “individual”, “subject”, “host” and “patient” are used interchangeably to refer to any subject for whom diagnosis, treatment, or therapy is desired or otherwise administered, particularly humans. Other subjects can include, but are not limited to, cattle, dogs, cats, guinea pigs, rabbits, rats, mice, horses, and the like.

Cells cultured by the methods described herein, e.g., chondrocytes, can be seeded into 3D scaffolds (matrices), and cultured in vitro in synovial fluid. This cartilage construct can be transplanted into the host site, for example, by direct injection or during a surgical procedure. As there are difficulties with introducing chondrocytes into joint cavities such as, for example, lack of adherence of the disperse chondrocytes to, for example, damaged cartilage, the 3D matrices provided herein would overcome problems encountered by previous attempts at such therapies.

Chondrocytes can be encapsulated in the alginate beads when transplanted into the host site. The alginate beads can be held in place by using, for example, other types of gels known to one of skill in the art. Chondrocytes can also be extracted from the alginate beads, as described herein, and encapsulated in other matrices, such as, for example, agarose, collagen, PEG or hyaluronan gels prior to implantation.

The system described herein for long-term 3D culturing of chondrocytes in synovial fluid provides significant improvement from previous culturing systems, which required, for example, a 2D environment and/or artificial medium (such improvements are evidenced, for example, by the observed reduction of cell death mediator, Caspase 3, expression in cells cultured with increasing ratios of synovial fluid in the culture medium). Because synovial fluid activity can be correlated to the combinatorial activities of many biochemical factors present in the fluid, the net effect of the synovial fluid on chondrocyte gene expression in the system described herein can be correlated to the severity of disease states and can be used to understand the intraarticular milieu before and after treatments, thereby allowing one of skill in the art to develop screens to identify factors and therapeutic agents that increase or otherwise modulate chondrocyte growth and cartilage production. The culturing system described herein is also useful for therapeutic purposes, as, for example, it allows for the use a subject's own synovial fluid for culturing autologous chondrocytes to regenerate articular cartilage, and to evaluate the response from individual subjects toward pharmaceutical therapies.

Cartilage in the adult mammal does not regenerate very efficiently in vivo; and as a result, osteoarthritis leads to irreversible cartilage loss accompanied by chronic pain and immobility (Centers for Disease Control and Prevention, MMWR Morb. Mortal Wkly. Rep., 56:423-5, 2007; Theis, K. et al., Arthritis Rheum., 57:355-63, 2007). Cartilage tissue engineering offers promising potential to regenerate and restore tissue function. This technology typically involves seeding chondrocytes into natural or synthetic scaffolds and culturing the resulting construct in a balanced medium over a period of time with a goal of engineering a biochemically and biomechanically mature tissue that can be transplanted into a defect site in vivo (Chung, C. and Burdick, J., Adv. Drug Deliv. Rev., 60:243-62, 2008; Glowacki, J., J. Rehabil. Res. Dev., 37:171-7, 2003; Chokalingam, K. et al., Tissue Eng. Part A, 15:2807-16, 2009; Butler, D. et al., J. Biomech. Eng., 122:570-5, 2000). Achieving optimal conditions for chondrocyte growth and matrix deposition is essential for the success of cartilage tissue engineering.

In the native joint cavity, cartilage at the articular surface of the bone is bathed in synovial fluid. This clear and viscous fluid provides nutrients to the avascular articular cartilage and contains growth factors, cytokines and enzymes that are important for chondrocyte metabolism (Goldring, M. and Goldring, S., J. Cell Physiol., 213:626-34, 2007; Zvaifler, N. and Firestein, G., Scand. J. Rheumatol. Suppl., 76:203-10, 1988). Synovial fluid also facilitates low-friction movement between cartilaginous surfaces, mainly through secreting two key components, hyaluronan and lubricin (Rhee, D. et al., J. Clin. Invest., 115:622-31, 2005; Campo, G. et al., Biochim. Biophys. Acta, 1812:1170-81, 2011). As tissue engineered cartilage is most often cultured in artificial media, such engineered cartilage is more likely to face tissue rejection if introduced into a host subject for therapy and/or other incompatibility of unsuitability issues. While artificial media are likely able to provide more defined conditions for studying chondrocyte metabolism, synovial fluid most accurately reflects the natural environment of which articular chondrocytes reside in.

“Treatment” refers to the administration of medicine or the performance of medical procedures with respect to a patient or subject, for either prophylaxis (prevention) or to cure or reduce the symptoms of the infirmity or malady in the instance where the patient is afflicted. Prevention of a disease or condition associated with depletion of cells that reside in synovial fluid, e.g., chondrocytes, is within the scope of treatment, e.g., for osteoarthritis or injury. The cells described herein can be used as part of a treatment regimen in therapeutically effective amounts. A “therapeutically effective amount” is an amount sufficient to decrease, prevent or ameliorate the symptoms associated with a medical condition. The present disclosure, for example, is directed to treatment using a therapeutically effective amount of a compound sufficient to treat a disease or condition associated with the depletion of cells that reside in synovial fluid.

The materials and methods described herein can be applied to any cell type, particularly those cell types whose natural environment is synovial fluid. For example, synoviocytes, meniscus cells and temporomandibular disc cells (TMJ) are examples of cell types that naturally reside in joint cavities bathed in synovial fluid. Synoviocytes come in two types: the macrophages, which increase significantly in number during arthritis; and the fibroblasts, which are responsible for secreting lubricating factors into the synovial fluid. The meniscus is a unique structure that is present in the knee joint, but not other joints. It is made of fibrocartilage, containing more fibrous collagens than articular cartilage does. TMJ is present in the joint cavity of the jaw bones and allows multi-directional chewing motions. The knee joint and the TMJ joints are the two joints in the body most prone to arthritis, thus the materials and methods described herein would be useful for therapies to treat, for example, arthritis in these joints, although treatment in other joints is also envisioned.

Indeed, synovial fluid has the advantage of being easy to obtain and store, and can often be regularly replenished by the body. Several groups have supplemented the culture medium with synovial fluid in growing human, bovine, rabbit and dog chondrocytes, but mostly used only low levels of synovial fluid (below 20%) (van de Lest, C. et al., Biorheology, 37:45-55, 2000; Saxne, T. et al., Coll. Relat. Res., 8:233-47, 1988; Lee, D. et al., Clin. Orthop. Relat. Res., (342):228-38, 1997; Schalkwijk, J. et al., Arthritis Rheum., 32:894-900, 1989; Schalkwijk, J. et al., Arthritis Rheum., 32:66-71, 1989; Joosten, L. et al., Agents Actions., 26:193-5, 1989; Schuerwegh, A. et al., Rheumatol. Int., 27:901-9, 2007; Hegewald, A. et al., Tissue Cell, 36:431-8, 2004; Xu, Q. et al., Tissue Cell, 41:13-22, 2009; Krüger, J. et al., J. Orthop. Res., 28:819-27, 2010; Steinhagen, J. et al., Tissue Cell, 42:151-7, 2010; Yang, K. et al., Tissue Eng., 12:2957-64, 2006; Skoog, V. et al., Scand. J. Plast. Reconstr. Surg. Hand Surg., 24:89-95, 1990; Nuver-Zwart, I., et al., J. Rheumatol., 15:210-6, 1988; Beekhuizen, M. et al., Arthritis Rheum., 63:1918-1927, 2011). While chicken, horse and human chondrocytes have been cultured in the medium with higher percentage of synovial fluid, these culture systems were two-dimensional (Rodrigo, J. et al., Am. J. Knee Surg., 8:124-9, 1995; van den Hoogen, B. et al., Br. J. RheumatoL, 37:671-6, 1998; Webb, G. et al., Osteoarthritis Cartilage, 6:167-76, 1998).

The materials and methods described herein offer the first long-term method of culturing human articular chondrocytes in a 3D system with a high percentage of synovial fluid (up to 100%). Described are results over a period of 21 days, however, one of skill in the art will recognize that such a period is sufficient to demonstrate longer term culturing under the same or similar conditions. This system provides the possibility of studying human chondrocytes in synovial fluid in a 3D setting, which can be further combined with two other important factors (oxygen tension and mechanical loading; Kook, S. et al., Cell Biol. Int., 32:871-8, 2008; Knobloch, T. et al., Crit. Rev. Eukaryot. Gene Expr., 18:139-50, 2008) that constitute the natural environment for cartilage to mimic the natural milieu for cartilage growth. The results described herein can also be used for assaying synovial fluid activity on chondrocytes and provide a platform for developing cartilage regeneration technologies and therapeutic options for arthritis.

The materials and methods described herein provide for long-term culturing methods for cells that reside in joint cavities and are bathed in synovial fluid in vivo. Examples include, but are not limited to, chondrocytes, synoviocytes, meniscus cells and TMJ. For

Chondrocytes are derived from mesenchymal stem cells and are found in joint cavities bathed in synovial fluid. They are the only cells found in cartilage. They produce and maintain the cartilaginous matrix, which consists mainly of collagen and proteoglycans. The organization of chondrocytes within cartilage differs depending upon the type of cartilage and where in the tissue they are found.

Synoviocytes, or synovial intimal cells, are responsible for the production of synovial fluid components, for absorption from the joint cavity, and for blood/synovial fluid exchanges, but their detailed structure and function as well as pathological changes remain unclear. Two types of synoviocytes, macrophagic cells (type A cells) and fibroblast-like cells (type B cells) have been identified. Type A synoviocytes are non-fixed cells that can phagocytose actively cell debris and wastes in the joint cavity, and possess an antigen-presenting ability. These type A cells are derived from blood-borne mononuclear cells. Type B synoviocytes are characterized by the existence of rough endoplasmic reticulum, and dendritic processes that form a regular network in the luminal surface of the synovial membrane.

Meniscus cells create the meniscus of the knee. The meniscus is a complex dense fibrous biologic structure with a design to provide stability. Collagenous components of the extracellular matrix (ECM) help provide tensile strength, and proteoglycan ECM components contribute to absorb shock and stress by the knee. The meniscus contains both a vascularized outer portion and an inner, avascular region. Appropriate function of the menisci is needed for normal knee biomechanical function. Aging and degeneration of the menisci result in tears being a common knee injury. Initial meniscal injury causes patient pain and disability, and further meniscal damage and/or loss is associated with degenerative joint changes ultimately leading to osteoarthritis.

Cells in the temporomandibular joint, the joint of the jaw frequently referred to as TMJ, are envisioned for culturing using the materials and methods described herein. There are two TMJs, one on either side of the jaw, working in unison. The unique feature of the TMJ is the articular disc. The disc is composed of fibrocartilagenous tissue (like the firm and flexible elastic cartilage of the ear), which is positioned between the two bones that form the joint. Cells of the TMJ articular joint provide stability and a cushion for the TMJ. Depletion of these cells lead can lead to, for example, osteoarthritis.

Cells cultured using the materials and methods described herein can be derived from a variety of sources. A subject in need of treatment, for example, can provide joint cells, e.g., chondrocytes, mesenchymal stem cells, TMJ disc cells, meniscus cells, etc., and/or synovial fluid. In such a situation, a subject's own cells can be cultured in the subject's own synovial fluid, thereby creating a highly tailored and personalized therapeutic cell line. If the cells cultured in this way are introduced back into the subject to treat, for example, a disease, disorder, condition or injury associated with depletion of joint cells and/or cartilage, there is an increased compatibility since the cells and culture medium are derived from the subject. Cells can also be derived from exogenous sources—either other subjects of the same species or from compatible but different species, depending on factors such as, for example, ease of obtaining the cells and cost of the cells. Synovial fluid used to culture such cells can be derived from the same source or from a suitably compatible source.

The cells cultured using the materials and methods described herein can be wild-type (unmodified) cells or genetically modified cells. As used herein, “genetically modified” refers to the introduction of an exogenous gene, the introduction of additional cop(ies) of an endogenous gene, the deletion of an endogenous gene, the expression modulation of an endogenous gene, or various combinations of these. Cells can be genetically modified to produce desirable characteristics. Chondrocytes, for example, with increased expression of IGF II tend to be more resistant to cell death, inflammatory-induced damage and senescence. Other genetic modifications, especially in situations where cells are being cultured for use as therapeutic agents, can produce changes to the cell that provide additional therapeutic benefits to a subject receiving the fells (e.g., expression of antibacterial agents, expression of immune suppression genes or immune response genes, or any other genetic trait as determined by one of skill in the art to be useful for the treatment to be tailored to a particular subject. It is therapeutically advantageous to introduce chondrocytes with phenotypic characteristics for example, that favor repair, that stimulate anabolic processes preferentially over catabolic, and/or that produce factors that inhibit catabolic pathways, or stimulate chondrogenesis.

The materials and methods described herein can be modified to customize, for example, therapies to particular joint and patient needs. Differences, for example, in the joint milieu that favor different therapeutic approaches, e.g., variation in the levels of proteases, cytokines, that predicate the type of intervention. There could be individual genetic variations, for example, that predicate responsiveness to different treatments and that can be identified using the materials and methods described herein as a way of personalizing the treatment approach or evaluating its effectiveness in an individual.

The cells cultured using the materials and methods described herein can be used for a variety of therapeutic purposes. Diseases, disorders, conditions or injuries that result from the depletion of joint cells and/or cartilage, for example, can be treated by the introduction of cultured cells into the environment where such depletion occurs. Examples of such diseases, disorders, conditions or injuries include, but are not limited to: arthritis, cartilage fractures, damage; meniscal tears, and cartilage damage due to other inflammatory disorders such as, for example, gout and rheumatoid arthritis.

Osteoarthritis, or Degenerative Joint Disease, is a common condition characterized by degradation of cartilage in joints, often accompanied by bone remodeling and bone overgrowth at the affected joints causing severe chronic pain and loss of mobility. The disease has many causes, but the end result is a structural degradation of joint cartilage and a failure of chondrocytes (cartilage-forming cells) to repair the degraded cartilage collagen matrix.

The present disclosure relates to a method of culturing cells that are suspended in a 3D matrix, such that the matrix allows for the exposure of the cells to a particular medium. The 3D matrix can, for example, be somewhat porous and allow for the surrounding media to disperse within the matrix, or it can be less porous, allowing only particular small molecule factors to pass into the 3D matrix. Alternatively, the matrix can allow for exposure of the cells on the outer surface of the matrix, with no exchange to the interior of the matrix. Examples of such matrices are known in the art, and include, but are not limited to, alginate matrices as described in the Example.

Alginate forms a semi-porous globular solid when exposed to a CaCl₂ solution. If an alginate solution is introduced drop-wise into a CaCl₂ solution, the resulting globular solid is a spherical bead. One of skill in the art would know how to vary the size and shape of the 3D matrix, by, for example, changing the volume of the drop introduced into the CaCl₂ solution, changing the distance from which the drop is introduced, etc. In certain embodiments, the methods described herein use alginate beads as 3D matrices that are spherical or substantially spherical, having average diameters of from about 0.1 mm to about 5 mm, from about 0.5 mm to about 3 mm, from about 1 mm to about 2.5 mm, or about 2 mm. As used herein, the term “about” refers to +/−20%, +/−10%, or +/−5% of a value. To enhance the structural integrity of the cartilage constructs, procedures can be modified for seeding chondrocytes into an alginate hydrogel matrix, including, for example, optimization of the length of time for bead formation and the media-changing procedure.

The methods described herein can also be used with other gel matrices. Other gel-related matrices include, for example, collagen, silk, hyaluronan, PEG, chitosan and agarose. In addition, chondrocytes can be seeded into porous scaffolds such as, for example, silk, collagen and polylactic acid.

The cells suspended or encapsulated in the 3D matrix are cultured in varying percentages of synovial fluid, with the greater percentage of synovial fluid more closely mimicking the cell's natural environment. As described, synovial fluid contains factors that affect cell growth and physiology. These factors, when in contact with the cells produce a cell more closely resembling the cell in situ. The 3D matrix can be adjusted such that cells encapsulated or suspended in the 3D matrix come into greater or lesser contact with the synovial fluid medium. One of skill in the art would know to change the size (thereby affecting the surface area, for example, of an alginate bead), shape or porosity of the 3D matrix to regulate the exposure of a cell to the surrounding medium. In additional, external mechanical mechanisms can be used to increase or decrease the amount of contact a cell suspended or encapsulated in a 3D matrix has with its surrounding environment, e.g., by agitation, shaking, rocking stirring etc.

Cells suspended or encapsulated in a 3D matrix as described herein can be cultured in a medium containing varying percentages (v/v) of synovial fluid, e.g., about 20%, greater than about 20%, about 30%, about 40%, about 50%, about 60%, about 70%, about 80%, about 90% or about 100%. The fact that the materials and methods described herein can, in some embodiments, allow for the culturing of cells entirely in synovial fluid allows, for the first time, cells to be cultured in vitro in an environment that mimics their natural environment in situ.

One of skill in the art will recognize that the use of 100% synovial fluid most closely mimics the natural milieu of cells that reside in joint cavities. That said, artificial of exogenous media can be mixed with synovial fluid as described in the Example. Although supplementing with other media would create a more artificial environment, one of skill in the art will recognize that conditions can be modified to produce desired results. For example, if the goal is to enhance tissue engineering, many other types of media can be supplemented to obtain the best quality cartilage, as would be determined by one of skill in the art.

The medium in which cells are cultured can include additional endogenous or exogenous factors. To study cartilage biology, for example, factors that are normally present in the synovial fluid can be added into the synovial fluid (such as cytokines and growth factors). In this case, the role of these factors can be evaluated in the normal growth environment of chondrocytes. The materials and methods provided herein provide a more appropriate setting in which to study the role of synovial fluid factors that can affect the development of cells that reside in joint cavities, e.g., chondrocytes. Using the materials and methods described herein, in fact, allows one of skill in the art to screen for factors that modulate the growth or differentiation of such cells.

To study cartilage repair or to develop cells that can be used in situ for cartilage repair or treatment of a disease or condition characterized by depletion of joint cavity cells, e.g., chondrocytes, many growth factors can be introduced into the synovial fluid during culturing. The effects of many growth factors on chondrocytes are typically determined first in in vitro cultures in the presence of artificial medium. However, once these factors are introduced into the joint cavity, they many have different effects as the environment is made up of synovial fluid. Therefore, one of skill in the at will appreciate the benefits of testing such factors in the cells and culture systems described herein.

The culturing methods described herein can be tailored to regulate exposure of suspended or encapsulated cells to synovial fluid by, for example, regulating or controlling the number and duration of media changes. Media, containing varying percentages of synovial fluid, can be changed, for example, about every 6 hours, about every 12 hours, about every day, about every 2 days, about every 3 days, about every 4 days, about every 5 days or about every 6 days. In addition, culture media can be exchanged constantly with constant circulation of media.

To further regulate exposure of suspended or encapsulated cells, a perfusion system can be used. When used in cartilage tissue engineering, a perfusion system actively forces nutrients to pass through the tissue. In such a system, cartilage constructs (3D scaffolds seeded with chondrocytes) are placed in a chamber filled with medium. A pump pushes the liquid from one end of the chamber to the other, providing the mechanical stimulation that allows nutrients to actively, not passively move through the tissues. As the cartilage tissue contains large amounts of extracellular matrix, cartilage is a rather dense tissue. Moving the media through cartilage actively helps chondrocytes to access the nutrients, thus, for example, allowing chondrocytes grow more healthily. Such motion mimics the movements during exercise (Alves da Silva, M. et al., J. Tissue Eng. Regen. Med., 5:722-32, 2010).

The materials and methods described herein can be used to evaluate the effect of potential therapeutic agents. Chondrocytes, for example, cultured as described herein, except that varying doses of a candidate therapeutic agent can be introduced into the medium in which the chondrocytes are cultured. Gene expression or particular genes of interest will be performed on chondrocytes cultured in the presence and absence of the potential target therapeutic agent. Chondrocytes from arthritis patients, for example, have different profiles of gene expression and phenotypes depending on diseases stages and severity. By identifying the gene expression profiles and determining the effect potential therapeutic agents have on gene expression will allow one of skill in the art to identify the efficacy of a therapeutic agent in treating, for example, arthritis. If an arthritic gene expression profile reverts to a more or less non-arthritic gene expression profile, then the target therapeutic agent is identified as a therapeutic agent effective for treating, for example, arthritis. In general, chondrocytes from osteoarthritis patients express lower levels of cartilage matrix genes and higher levels of genes associated with cartilage destruction (such as proteinases). The diseased chondrocytes also express higher levels of inflammatory cytokines and are in a more senescent state.

Synovial fluid is one of the major components that constitute the natural environment in the joint cavity, where articular chondrocytes reside. While the results described herein were obtained by culturing normal human chondrocytes in synovial fluid derived from patients with osteoarthritis, this method can be used synovial fluid from an otherwise healthy person or other healthy vertebrate animals. Because synovial fluid activity can be correlated to the combinatorial activities of many biochemical factors present in the fluid, the net effect of the synovial fluid on chondrocyte gene expression in the systems described herein can be correlated with the severity of disease. In one embodiment, the systems and methods described herein allow for the use of a subject's own synovial fluid for culturing autologous chondrocytes to regenerate articular cartilage, thereby tailoring therapies to a particular individual. To this end, this system may provide insights into studying the biomechanical forces acting through synovial fluid on engineered cartilage as well (Wright, V. and Dowson, D., J. Anat., 121:107-18, 1976).

TABLE
Specific Reagents and Equipment

| Name of the Reagent | Company | Catalogue Number | Comments |
|---|---|---|---|
| Alginate (Alginic | Sigma | A2158-250G | 2.4% solution |
| Acid sodium salt) | | | stored at 4° C. |
| Calcium Chloride | J. T. Baker | A19339 | |
| Dihydrate, Granular | | | |
| Chondrogenic Growth | Lonza | CC-3156 | |
| media | | (media base) | |
| | | CC-4409 | |
| | | (supplement) | |
| Chondrogenic | Lonza | CC-3226 | |
| Differentiation Media | | (base media) | |
| | | CC-4408 | |
| | | (supplement) | |
| Human articular | Lonza | CC-2550 | |
| chondrocytes | | | |
| Dapi (4′,6-Diamino-2- | Sigma-Aldrich | D9542 | |
| phenylindole | | | |
| dihydrochloride) | | | |
| RNeasy mini kit | Qiagen | 74104 | |
| (for RNA extraction) | | | |
| PCR reagents: SYBR- | Quanta | 95053-500 | |
| green | | | |
| 12 ml syringe | Tyco-Kendall-Monoject | 512852 | |
| 22-Gague Hypodermic | Tyco-Kendall-Monoject | 8881 | |
| Needle | | | |
| Microscope | Olympus | IX71 | |
| Platform rocker | Thermoscientific | Vari-mix | |
| | thermolyne | | |
| Primers sequences | | | |
| Collagen IIa-forward | 5′-TTC ATC CCA CCC TCT CAC | | |
| | AGT-3′ | | |
| | (SEQ ID NO: 1) | | |
| Collagen IIa-reverse | 5′-CCTCTGCCTTGACCCGAA-3′ | | |
| | (SEQ ID NO: 2) | | |
| MMP13-forward | 5′-TGT GCC CTT CTT CAC ACA | | |
| | GAC ACT-3′ | | |
| | (SEQ ID NO: 3) | | |
| MMP13-reverse | 5′-GAG AGC AGA CTT TGA GTC | | |
| | ATT GCC-3′ | | |
| | (SEQ ID NO: 4) | | |
| Caspase 3-forward | 5′-TCA TTA TTC AGG CCT GCC | | |
| | GTG GTA-3′ | | |
| | (SEQ ID NO: 5) | | |
| Caspase 3-reverse | 5′-TGG ATG AAC CAG GAG CCA | | |
| | TCC TTT-3′ | | |
| | (SEQ ID NO: 6) | | |

### EXAMPLE

### A 3D System for Culturing Human Articular Chondrocytes in Synovial Fluid

Human articular chondrocytes were encapsulated in alginate beads using a modified manufacture-suggested encapsulation protocol (Lonza; and Guo, J. et al., Connect. Tissue Res., 19:277-97, 1989). Using these 3D constructs, a system for culturing cells in a culture medium containing varied percentages of human synovial fluid was developed. The cells in these 3D constructs were examined for cartilage gene expression. The steps for preparing the cells were as follows:
1. Human articular chondrocytes (HAC) prepared for three-dimensional (3D) encapsulation
   a) One vial (1 mL) of human articular chondrocytes (HAC) (Lonza) (Passage 2) thawed in 37C water bath for 1 min;
   b) Mixed with 1 mL of chondrocyte growth media (Lonza) and centrifuged at 3000 rpm for 3-5 min to collect the cells. Discarded the supernatant;
   c) Resuspended cell pellet in chondrocyte growth medium (Lonza);
   d) Plated cells on a 10 cm tissue culture plate (BD Biosciences), and cultured in chondrocyte growth media (Lonza) until the cells were confluent. HACs used at a passage number no higher than 3 or 4; and
   e) Washed HACs on the plate with 155 mM NaCl instead of the standard PBS, followed by trypsinzation to collect cells for alginate bead encapsulation. Once cells detached, they were spun spin down the cells at 3000 rpm for 5 min.
2. Encapsulation of HACs into 3D Beads

Cells from 1) were resuspended in 1.2% alginate solution (Sigma) at a density of 8×10⁵ cells/mL. Cell numbers were determined prior to encapsulation, using a standard cell counter. The suspension was mixed well to ensure even distribution of the cells in the beads.
a) Pipetted the HAC/Alginate solution mixture into a 12 mL syringe attached to a 22-gauge needle (Tyco healthcare, Inc.);
b) Meanwhile, a 50 mL beaker was prepared with 102 mM CaCl₂ at a volume 5 times that of the HAC/alginate solution. The solution was stirred slowly (at approximately 150 rpm);
c) A syringe tip was held about 6 inches above the surface of the CaCl₂ solution and the HAC/alginate mixture was added to the CaCl₂ solution dropwise. In general, the resulting alginate beads were about 2 mm in diameter with an encapsulation density of approximately 10⁴ cells/bead. Note: The height of the syringe over the CaCl₂ solution can be used to control the size and shape of the beads; a shorter distance will result in tear-drop shaped instead of spherical shaped beads, causing uneven mechanical integrity and cell encapsulation;
d) The bead were stirred in the CaCl₂ solution for 20-25 min (while other alginate encapsulation protocols indicate a much shorter incubation time of 10 min, a longer incubation time with the CaCl₂ solution was found to enhance the integrity of the beads);
e) The CaCl₂ solution was removed and the beads were washed 2-3 times in 2-3 volumes of NaCl, and then once in chondrocyte differentiation medium (while other alginate bead encapsulation protocols involve using a filter for the above washing procedures, it was found that the alginate beads often become trapped in the filter and dry out, thus reducing the efficiency of encapsulation. It was found to be most efficient to allow the beads to settle to the bottom of the beaker before discarding the solution); and
f) Used a standard spatula to transfer the beads into culture dishes (12 beads per 3 cm well were usually cultured for 2 days in chondrocyte growth medium to allow chondrocytes to adjust to the encapsulation process in the medium they were grown in before switching to chondrocyte differentiation medium (Lonza) with synovial fluid).
3. Chondrocytes were cultured in synovial fluid culture medium.
   a) Fresh synovial fluid can be obtained from an outpatient clinic (synovial fluid used herein was obtained from Tufts Medical Center). The synovial fluid can be transferred in 15 ml falcon tubes to the laboratory, and immediately centrifuged at 3000 rpm for 15 min to remove cell debris. Cell-free synovial fluid was aliquotted into 1.5 mL microcentrifuge tubes to avoid repeated freeze-thawing. Supernatant can be stored at −80 C until use;
   b) Prior to culturing, synovial fluid was mixed with chondrocyte differentiation medium (CDM, Lonza) at varying volumetric ratios, with a constant concentration of 100 mM ascorbic acid and 9 mM CaCl₂ (this trace amount of CaCl₂ was to ensure the integrity of the alginate beads);
   c) The plate was placed on a rocking platform in a 37 C incubator (rocking frequency: about 75 times/min) to help nutrient distribution within the alginate beads, and to reduce clumping of the beads. This is useful as chondrocytes can be cultured for an extended period of time (up to 4 weeks or longer). Note: While chondrocyte growth and differentiation media (Lonza) contain two commonly used antibiotics gentamycin and amphotericin, antibiotics were not supplemented when varying percentages of synovial fluid was used for chondrocyte culturing-no contamination was ever observed; and
   d) Media mixtures were changed every 2-3 days (although the viscosity of synovial fluid has been a major hurdle in culturing chondrocytes encapsulated in alginate beads during long-term cultures, it was observed to be most effective to dilute synovial fluid-supplemented medium with 102 mM CaCl₂ (50% V/V), which also strengthens the integrity of the alginate beads. In this way, the medium can slowly be removed with a pipette, without damaging the beads.).
4. HACs were harvested in alginate beads for gene expression analysis.

Special care must be taken when harvesting the HACs from the alginate beads for gene expression analysis.
a) The alginate beads were washed 3 times in 102 mM CaCl₂ for about 5 min each time;
b) Chondrocytes were retrieved by adding 55 mM NaCitrate at a volume 4-5 times that of the beads (it is important that the alginate beads are totally immersed in the NaCitrate solution), and the mixture was shaken or rocked for 20-30 min;
c) The cells were spun down, and the supernatant was discarded; and
d) For RT-PCR analysis, the cell pellet was resuspended in cell lysis buffer (Qiagen RNA isolation kit), and RNA was isolated and purified according to standard protocol.
5. HACs fixed in alginate beads for histological analysis.

Special care has to be taken to harvest the HACs from the 3D beads for histological analysis.
a) The alginate beads were washed 3 times in 102 mM CaCl₂ for about 5 min each time (to completely wash away the viscous synovial fluid, it was most effective to wash with chondrocyte differentiation medium (Lonza) overnight, rocking in a 37 C incubator (rocking frequency: about 75 times/min). The chondrocyte growth medium was used to ensure chondrocyte survival in this prolonged wash and to allow maximum penetration of the fixing agent (such as paraformaldehyde)); and
b) The beads were fixed in 70% ethanol (alternatively, one can use a 4% paraformaldehyde solution) overnight, allowing for subsequent histological analysis (briefly, for perform Dapi staining, alginate beads were incubated with Dapi solution (500 ng/mL) for 1 hr under gentle rocking conditions, and washed for 3 times with 102 mM CaCl₂ for 15 min each time. Dapi images can then be viewed, for example, under a fluorescent microscope. The beads can also be sectioned for alcian blue and H&E staining.).

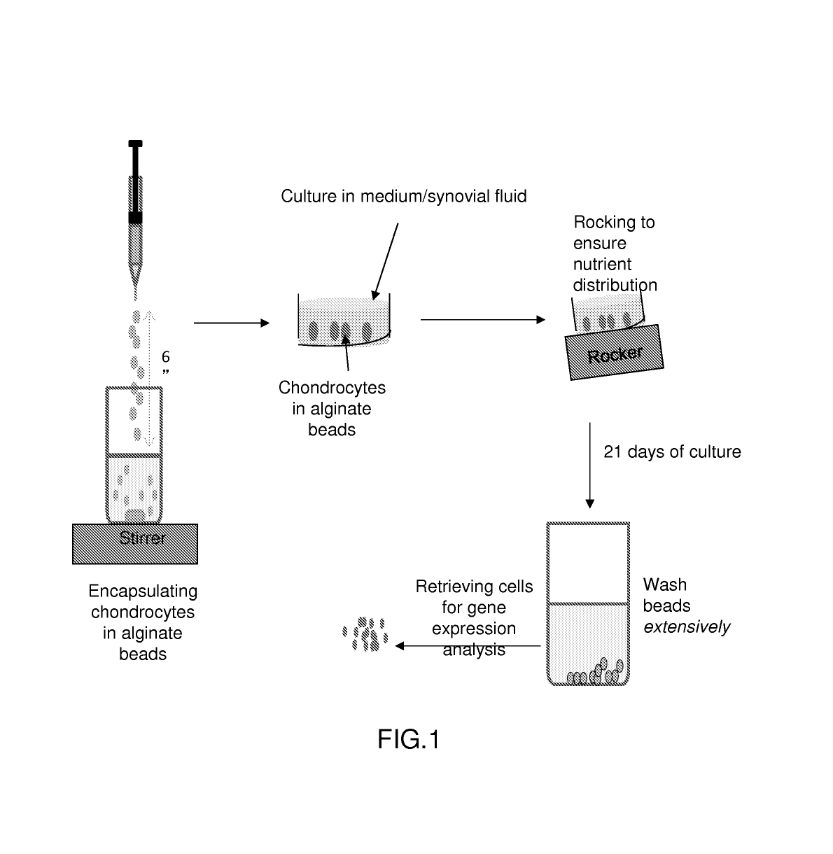

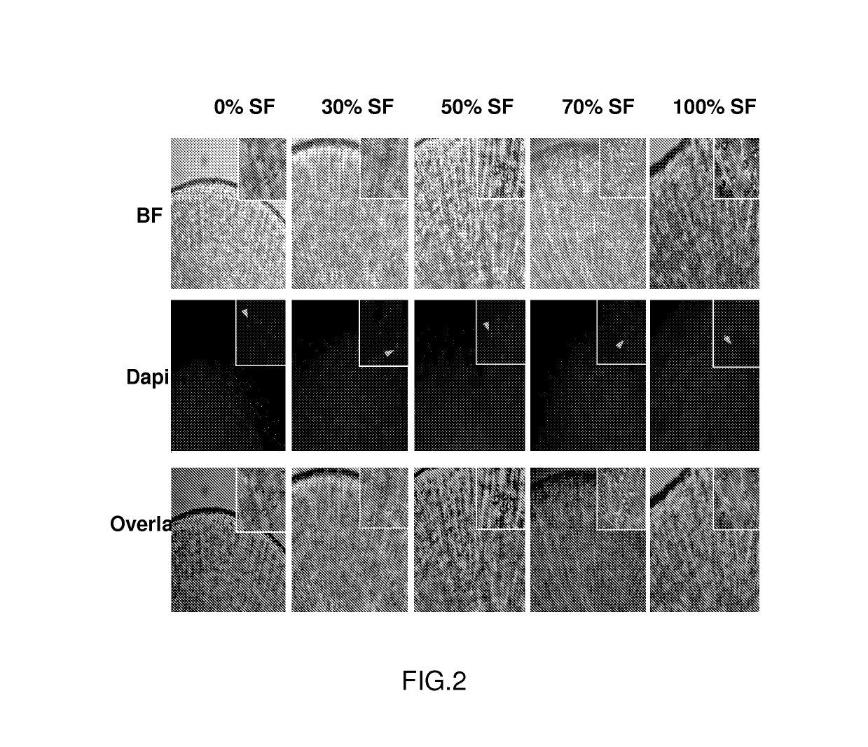

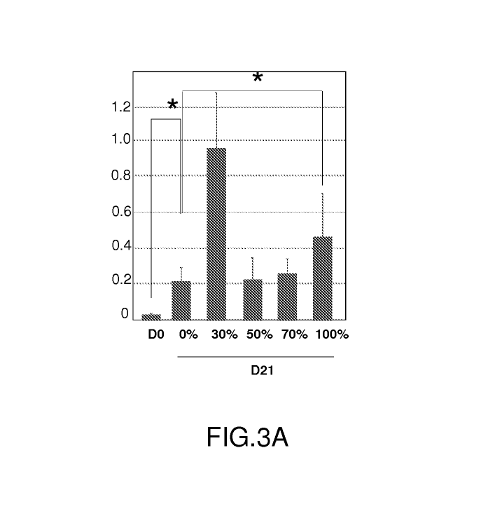

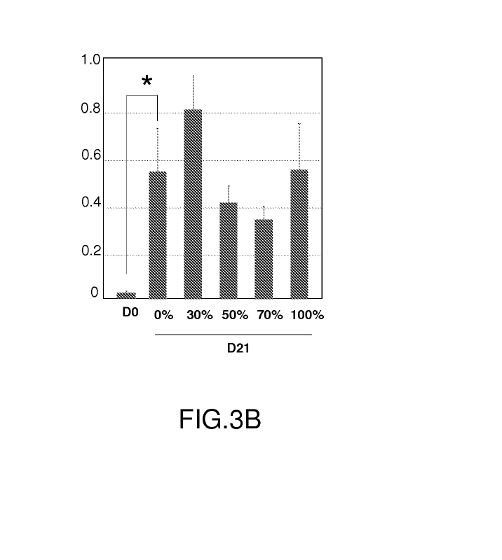

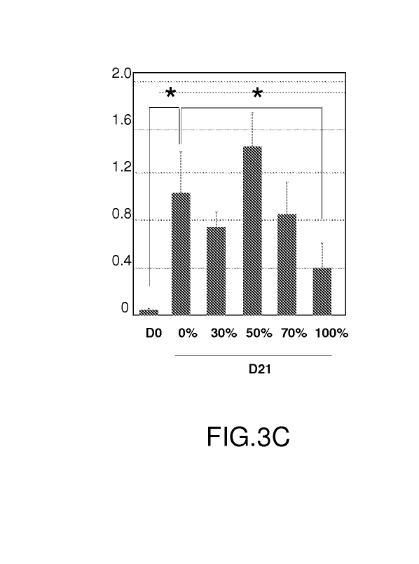

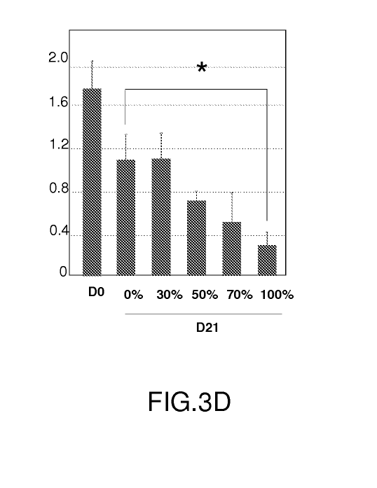

The 3D culturing method for human chondrocytes in high percentages of synovial fluid is depicted in the schematic diagram shown in FIG. 1. After human chondrocytes were encapsulated in alginate beads, they were allowed to grow in medium supplemented with varying ratios of synovial fluid. Because of the viscosity of the synovial fluid, it is useful to culture chondrocytes under constant rocking conditions to prevent the clumping of the cartilage constructs and to ensure even distribution of the nutrients. It is also useful to wash the alginate beads extensively before retrieving the chondrocytes, so that the fixing or lysis buffer can penetrate the beads (FIG. 1). Bright field (BF) images of chondrocytes in the alginate beads are shown in FIG. 2. Dapi staining was performed to confirm uniform distribution of the cells within the beads (FIG. 2). At the end of the 21-day culture period, gene expression analysis was performed by qRT-PCR. The reference gene, GAPDH, was used for normalization for all PCRs, as it was determined to be one of the most reliable reference genes for qPCR analysis on chondrocytes (Toegel, S. et al., BMC Mot Biol., 8:13, 2007). An example is shown in FIG. 3, where the results from human articular chondrocytes cultured in synovial fluid pooled from six patients with osteoarthritis were analyzed. Consistent with the fact that the chondrocytes from Lonza have been expanded in 2D cultures, which would inevitably lead to de-differentiation (Lin, Z. et al., J. Orthop. Res., 26:1230-7, 2008), Day 0 chondrocytes expressed minimal levels of cartilage matrix genes (FIG. 3). 3D culturing of chondrocytes with chondrocyte differentiation medium (Lonza) or medium supplemented with synovial fluid significantly increased cartilage gene expression of collagen II, aggrecan and MMP13, which indicates chondrocyte re-differentiation by Day 21 (FIG. 3; Goessler, U. et al., Int. J. Mot Med., 16:509-15, 2005). Increasing the percentage of synovial fluid in the media resulted in comparable levels of cartilage matrix markers Collagen II and aggrecan mRNA expression (FIGS. 3A and 3B). Furthermore, chondrocytes cultured in 100% synovial fluid even exhibited a decrease in cartilage degrading enzyme MMP13 mRNA expression as compared with those cultured in medium alone (FIG. 3C). Interestingly, the expression level of cell death indicator Caspase 3 gradually decreased with increasing ratios of synovial fluid, suggesting that synovial fluid culturing has led to decreased apoptosis levels (FIG. 3D). Therefore, our results show that culturing human articular chondrocytes in high levels of synovial fluid in a 3D setting is a feasible technology, while conferring particular advantages over 2D culturing systems or culturing systems that rely on otherwise low levels of synovial fluid.

### Other Embodiments

Other embodiments will be evident to those of skill in the art. It should be understood that the foregoing detailed description is provided for clarity only and is merely exemplary. The spirit and scope of the present disclosure are not limited to the above examples, but are encompassed by the following claims. The contents of all references cited herein are incorporated by reference in their entireties.

What is claimed is:

1. A method of culturing mammalian cells that reside in a joint cavity in vivo, comprising:
a) suspending one or more isolated cells in a three-dimensional matrix; and
b) incubating the suspended cells in a culture medium comprising at least about 20% synovial fluid under conditions suitable for chondrocyte division.

2. The method of claim 1, wherein the mammalian cells are selected from the group consisting of: chondrocytes, synoviocytes, meniscus cells and temporomandibular disc cells.

3. The method of claim 2, wherein the cells are chondrocytes.

4. The method of claim 3, wherein the chondrocytes are obtained commercially or derived directly from a mammalian subject.

5. The method of claim 3, wherein the mammalian chondrocytes are human chondrocytes.

6. The method of claim 1, wherein the culture medium comprises at least about 20% synovial fluid, 30% synovial fluid, 40% synovial fluid, 50% synovial fluid, 60% synovial fluid, 70% synovial fluid, 80% synovial fluid, 90% synovial fluid or 100% synovial fluid.

7. The method of claim 1, further comprising one or more incubations in a culture medium comprising at least about 20% synovial fluid.

8. The method of claim 7, wherein the culture medium is changed at least about every two or three days.

9. The method of claim 1, wherein the three dimensional matrix comprises alginate.

10. The method of claim 9, wherein the three dimensional makes forms a spherical bead about 2 mm in diameter.

11. The method of claim 1, wherein the culture medium is supplemented with CaCl₂.

12. The method of claim 1, further comprising treating the suspended cells with sodium citrate.

13. The method of claim 1, wherein the one or more suspended cells have been genetically modified.

14. A method of treating an injury, disease or disorder characterized by chondrocyte depletion comprising administering to a subject afflicted with an injury, disease or disorder characterized by chondrocyte depletion, an effective amount of cultured chondrocytes, wherein the chondrocytes are cultured in at least about 20% synovial fluid and wherein the chondrocytes are suspended in a three-dimensional matrix during culturing.

15. The method of claim 14, wherein the synovial fluid used to culture the chondrocytes is derived from the subject afflicted with the disease or disorder.

16. The method of claim 14, wherein the culture medium comprises at least about 20% synovial fluid, 30% synovial fluid, 40% synovial fluid, 50% synovial fluid, 60% synovial fluid, 70% synovial fluid, 80% synovial fluid, 90% synovial fluid or 100% synovial fluid.

17. The method of claim 14, wherein the three dimensional matrix comprises alginate.

18. The method of claim 17, wherein the three dimensional makes forms a spherical bead about 2 mm in diameter.

19. The method of claim 14, wherein the method is used to treat osteoarthritis.

20. The method of claim 14, wherein the cultured chondrocytes are removed from the three dimensional matrix prior to administering them to the subject.

21. The method of claim 14, wherein the three-dimensional matrix comprises alginate.

22. The method of claim 21, wherein the cultured chondrocytes are removed from the three dimensional matrix prior to administering them to the subject, said removal comprising washing the cells suspended in the matrix with sodium citrate.

23. The method of claim 14, wherein the cultured chondrocytes administered to the subject have been genetically modified.

24. A mammalian chondrocyte cultured by a method comprising:
a) suspending one or more chondrocytes in a three-dimensional matrix; and
b) incubating the suspended chondrocytes in a culture medium comprising at least about 20% synovial fluid under conditions suitable for chondrocyte division.

25. The chondrocyte of claim 24, wherein the one or more suspended chondrocytes have been genetically modified.

26. The chondrocyte of claim 24, wherein the three-dimensional matrix comprises alginate.

27. The chondrocyte of claim 26, wherein the chondrocyte is removed from the three-dimensional matrix by washing in sodium citrate.

28. A method of screening compounds for treating a disease or condition characterized by a depletion of cells in a joint cavity or loss of function of cells in a joint cavity, comprising:
a) suspending one or more cells in a three-dimensional matrix, wherein the one or more cells are isolated from a subject with a disease or condition characterized by a depletion of cells in a joint cavity or loss of function of cells in a joint cavity;
b) incubating the suspended cells in a culture medium comprising at least about 20% synovial fluid under conditions suitable for chondrocyte division in the presence and/or absence of a test compound; and
c) determining a gene expression profile for one or more genes indicative of a disease state or condition, wherein a gene expression profile of the cells cultured in the presence of the test compound that is more similar to the gene expression profile of a healthy individual than to the gene expression profile of a subject afflicted with the disease or condition indicates the test compound is effective in treating the disease or condition.

29. The method of claim 28, wherein the one or more isolated cells are chondrocytes.

30. The method of claim 28, wherein the disease or condition is osteoarthritis.
